(19) United States
(12) Patent Application Publication
(10) Pub. No.: US 2006/0167422 A1
(43) Pub. Date: Jul. 27, 2006

(54) Heat Shrink Scleral Band With Custom-Made Buckle For Retinal Detachment Surgery

(75) Inventors: Mohsen Shahinpoor, Albuquerque, NM (US); David Soltanpour, New York, NY (US); Parsa Shahinpoor, Albuquerque, NM (US)

Correspondence Address: MOHSEN SHAHINPOOR, 909 VIRGINIA, NE, SUITE 205, ALBERQUERQUE, NM, 87108, US

(73) Assignee: mohsen shahinpoor, albuquerque, NM (US)

(21) Appl. No.: 10/707,662

(22) Filed: Dec. 30, 2003

(51) Int. Cl. A61M 35/00; A61F 2/16

(52) U.S. Cl. 604/294; 606/27; 623/6.63

(57) ABSTRACT

Discussed is a method and apparatus of correcting retinal detachments by heat-shrinking a specially designed scleral band equipped with a snap-on custom-made buckle for scleral indentation over the retinal tear region. The heat shrink band is made from a heat shrink material such as a polymer or an alloy and is equipped with a custom-made buckle to perfectly fit the topological geometry of the retinal detachment region over the underlying choroid and the sclera, the conjunctiva and within the Tenon's space wherein the band is heat shrunk to create a perfect buckle over the retinal tear region to reattach the retina to the choroid and cause absorption of the subretinal fluid. Conventional, laser, microwave, Joule or radio-frequency (RF) heating means are also discussed.

### BACKGROUND OF INVENTION

1. Field of the Invention

This invention relates to retinal reattachment surgery, and more particularly to a method which uses heat induced shrinkage of a specially designed scleral band made with heat shrink biocompatible materials equipped with a custom-made buckle to facilitate retinal reattachment.

2. Prior Art

The function of retina is to transmits image signals through the fibers of the optic nerve to the brain. To function properly, the retina must be in contact with the choroid, from which it receives a significant portion of the oxygen and glucose required for its normal nourishment.

If the retina is detached from the choroid, it is therefore no longer able to accomplish its function. Retinal detachment is typically the result of subretinal fluid that has permeated between the retina and the choroid through one or more either round holes or horseshoe shaped holes in the structure of the retina. The permeated sub-retinal fluid causes portions of the retina to break away from the choroid. Patients suffering from retinal detachment observe some deterioration in their visual acuity and field of vision. Treatment of retinal detachment requires occlusion and repair of the ruptures of the retina. In the prior art, occlusion is accomplished by suturing permanent silicone implants to the outer wall of the sclera. The implants cause a buckling or bulging in the sclera toward the area of the retinal holes by means of a buckle placed under the silicone band or implant right over the retinal tear area. The purpose of the buckle is to create enough scleral indentation (bump) for the choroid to approximate the detached retina following subretinal fluid drainage. Most buckling surgical procedures require surgical incisions and dissections to position the implant that has to be sutured to the sclera in order to create a stable and adequate buckling effect. The implant, also known as an explant, may be made, for example, of several forms of silicone (solid, sponge or foam) rubber. The buckling in the sclera causes the sclera and its internally contiguous layer, the choroid, to move inward and occlude the holes of the detached retina. After this occlusion occurs, sub-retinal fluid is reabsorbed, allowing the retina to settle back into position, in contact with the choroid. In order to induce a scarring reaction and augment the adhesiveness of the retina to its normal position in the areas in which it was previously detached, laser heating, diathermy, cryotherapy, or other forms of laser or radio frequency heating therapy are also applied to the area of the retinal rupture. Retinal detachment is a serious eye disease that can lead to blindness. The retina may detach along an edge as the result of trauma, or as the result of a tear allowing fluid to leak underneath the retina and separate the retina from the underlying choroid. Retinal detachment can be treated by means of a scleral buckle, a silicone band that encircles the eye and indents the wall of the eye inward against the retina by means of a buckle over the retinal rear region. Alternatively, the vitreous humor may be replaced in whole or in part with a tamponade, a heavy liquid such as silicone oil or gas intended to flatten the retina against the choroid. The reader is referred to the classical patent disclosure, by Lincoff, entitled: ophthalmological appliance, U.S. Pat. No. 4,299,227 issued Nov. 10, 1981 in which a method of correcting retinal detachments through a small conjunctival incision is disclosed wherein an expandable member is inserted into Tenon's space, the member is expanded, to form an indentation in the eye, and left in place until the subretinal fluid has absorbed and the retina has reattached. Refojo, in U.S. Pat. No. 4,453,776, entitled: hydrogel implant article and method, issued Jun. 5, 1984 discusses a hydrogel implant article of significant softness, pliability and elasticity when dry as well as when wet consists of a random cross-linked copolymer of 2-hydroxyethyl acrylate and an alkyl acrylate. The acrylate hydrogel article is useful, in one instance, as the material to produce a scleral buckle in retinal detachment surgery. U.S. Pat. No. 5,286,261 to Roizenblatt, entitled: inflatable balloon for treatment of retinal detachment, issued Feb. 15, 1994 discusses an inflatable balloon for use in the treatment of retinal detachments. The balloon has a hollow elliptic-shaped body, a solid base, and a solid cylindrical stem. The balloon is inserted into Tenon's space by inserting a fine blunt-ended needle longitudinally through the solid base and stem of the balloon. Ren, et. al. in U.S. Pat. No. 5,423,800, issued Jun. 13, 1995 and entitled: laser scleral buckling method and instruments therefore, discusses a method of retinal re-attachment including the steps of providing an optical fiber probe assembly; providing a laser system coupled to the optical fiber probe assembly for delivering at least one laser light beam to the optical fiber probe, delivering the laser light beam from the laser system through the optical fiber probe assembly to the sclera at the locus of the retinal break area thereby to produce localized scleral shrinkage to buckle the sclera so as to relax the retinal break area. An apparatus is also provided to employ the method. Further, Ren, et al. in U.S. Pat. No. 5,688,264 issued Nov. 18, 1997 and entitled: Laser treatment for retinal detachment, teaches the use of an endoprobe including a tubular element having a proximal end and a distal end. U.S. Pat. No. 6,135,118, entitled treatment with magnetic fluids, issued to Dailey on Oct. 24, 2000, teaches a method for treating retinal detachment in an eye which includes inserting a magnetic fluid into the vitreal chamber of the eye and applying a magnetized scleral buckle, to the eye to treat retinal detachment. Furthermore, Dailey in U.S. Pat. No. 6,547,714 entitled: Magnetized scleral buckle for use with silicone magnetic fluids in the treatment of retinal diseases, which was issued on Apr. 15, 2003 and U.S. Pat. No. 6,612,311 entitled: Treatment with magnetic fluids, discusses a new method to treat retinal detachment by combining usage of a magnetic fluid tamponade with a magnetized flexible scleral buckle, which effects tamponade of the retina without interfering with vision.

The current procedures for treating retinal detachment, requires the patient to undergo general anesthesia. The surgery also demands a substantial amount of time. Moreover, because the procedure requires extensive surgical manipulation of ocular tissue, the patient is likely to suffer from pain and ocular edema in the post-operative period. Furthermore, substantial dexterity is needed in order to suture the solid implant to the patient's sclera. Therefore, a need exists for a user friendly heat-shrink buckle band with custom-made buckle for use in retinal detachment surgery that does not require extensive sutures and can be easily placed around the sclera. Prior art developments in correcting retinal detachment with scleral buckling are described in Sutureless Scleral Buckling by G. A. Calabria, R. C. Pruett, M. F. Refojo, and C. L. Schepens, Archives of Ophthalmology, May 1970, Vol. 83, pp. 613-618.

The closure of retinal breaks leading to detachment often requires a degree of scleral indentation which, if attempted in one operation, would require drainage of sub-retinal fluid to preclude raising the internal pressure of the eye to an unacceptably high level. The drainage procedure has well known risks. Therefore, it is desirable to accomplish the total required indentation over a significant period of time rather than in one operation. The heat-shrink band allows the surgeon to gradually tighten the buckle, and indeed this constitutes one aspect of the invention. When buckling is accomplished relatively slowly, the intraocular drainage mechanism can compensate and undesirably high pressures within the eye are avoided. It is also desirable for the implant to have the ability to assume configurations intermediate the initial and final ones, to permit gradual indentation of the eye to the final desired shape for closing the retinal break. The heat delivery systems to cause the heat-shrink polymeric band to shrink is generally delivered by a continuous-wave (cw) Argon, a cw Krypton, YAG (Yttrium Aluminum Garnet) or a cw diode laser, conventional hot tip means, or radio frequency (RF) heating means, etc.

Accordingly, a need exists to provide a simplified and more advantageous and user-friendly surgical procedure of retinal reattachment using heat-shrink bands with custom-made buckles.

### SUMMARY OF INVENTION

One object of the present invention is to provide a heat-shrink band with a custom-made buckle for treatment of retinal detachments that does not require extensive suturing and can be done in a number of stages.

Another object of the present invention is to provide a specially designed heat-shrink buckle band for treatment of retinal detachments in which the amount and degree of retinal buckling is easily controlled during surgery.

A still further object of the present invention is to provide a custom-designed heat-shrink band for treatment of retinal detachments that does not require the patient to undergo general anesthesia.

Another object of the said invention is to provide a heat shrink band with controllable custom buckle to fit the exact geometry of the retinal tear for the treatment of retinal detachments.

Another object of the present invention is to provide a method which simplifies retinal reattachment surgery, thus, reducing surgical time and minimizing patient trauma.

These and other objects of the present invention are achieved by a heat-shrink band and custom-made buckle. The heat-shrink band equipped with a custom-designed buckle that perfectly fits the geometry of the retinal tear is inserted into Tenon's space, near the detached retinal portion where the retinal holes are located. The band is then heated by means of a hot tip, optical fiber-delivered laser, RF or other conventional means in order to achieve the desired amount of tension in the band and indentation in the sclera. The heat shrinking procedure may be done in stages so that in subsequent visits to the surgeon no open surgery is needed but only transcutaneous heating and shrinking of the band is performed to gradually drain the sub-retinal fluids and perfectly cover the detached area and have the retina reattach to the choroids.

These explanations and other objects of the said invention will become apparent from the study of the drawings and the following description of the preferred embodiments of the present invention.

### BRIEF DESCRIPTION OF DRAWINGS

FIGS. 1 (a), (b), (c), (d), and (e) depict embodiments of the progressive stages of heat-shrinking of the scleral retinal detachment band with a custom-designed buckle.

FIGS. 2 (a), (b) and (c), Various embodiments of the heat-shrink band with a large custom-designed flat-top hemi-spherical buckle.

FIGS. 3 (a), (b) and (c), Various embodiments of the heat-shrink band with a large custom-designed hemi-spherical buckle.

FIGS. 4 (a), (b) and (c), Various embodiments of the heat-shrink band with a small custom-designed hemi-spherical buckle.

FIGS. 5 (a), (b) and (c), various embodiments of the heat-shrink band with a custom-designed cylindrical buckle.

FIGS. 6 (a), (b) and (c), Various embodiments of the heat-shrink band with a flat-top custom-designed oval-shaped buckle.

FIGS. 7 (a), (b) and (c), Various embodiments of the heat-shrink band with a custom-designed irregular shape buckle.

### DETAILED DESCRIPTION

In accordance with the invention, it has been found that low temperature heat-shrink polymers such as medical grade polyurethane or polyolefin will be appropriate for the heat shrink band. It is, however, understood that non heat-shrink polymers such as silicone which is currently used for normal buckle surgery can also be used with custom-designed buckle in the present invention. This choice stemmed from the view that the silicone implants have been used for buckle surgery for almost three decades now with no adverse effect and certainly redesigning them with custom-designed buckle according to the present invention offers advantages already. Complementary to this view, this invention provides a medical grade heat-shrink polymeric material that functions well as implants without exhibiting these supposed shortcomings and, further, that have advantages over prior materials.

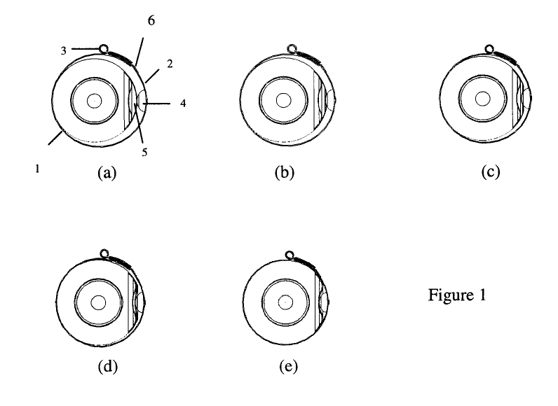

FIGS. 1 (a), (b), (c), (d), and (e) depict embodiments of the progressive stages of heat-shrinking of the scleral retinal detachment band with a custom-designed buckle. Note that in this figure, number 1 denotes the heat shrink band and eye assembly. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area 5. Initially the band is placed by the surgeon in a semi-tight manner as shown in FIG. 1(a). FIGS. 1(b) through 1(e) then depicts the sequences of heating the heat shrink band to shorten its circumferential length and to increase its tension to the desired tension necessary to indent the sclera over the retinal tear region 5, as shown in FIG. 1(e). Because the heat shrink band shrinks uniformly with heat the custom made buckle will approach the retinal tear area and gradually indent the sclera inward to reattach the retina to choroid.

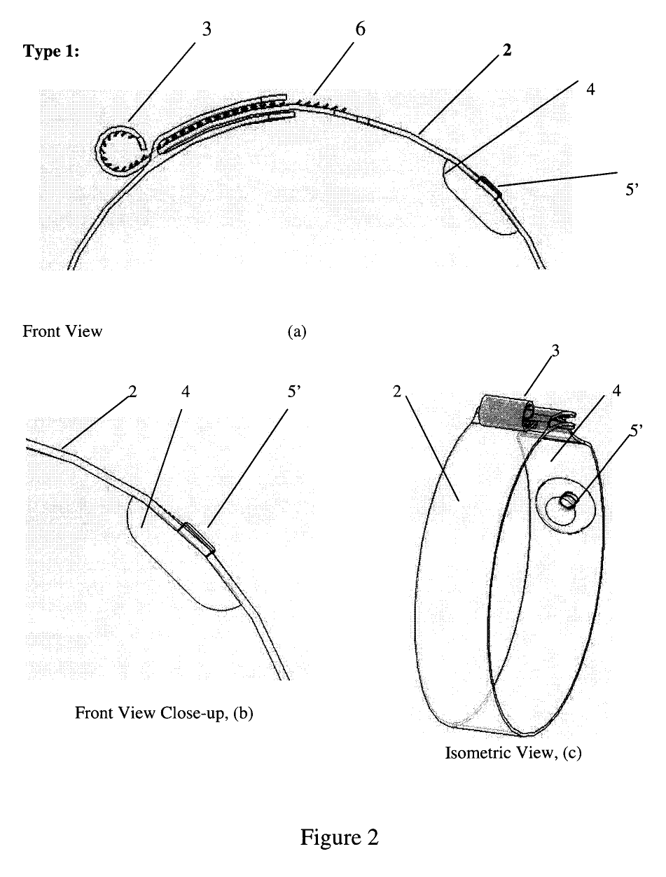

FIGS. 2(a), 2(b) and 2(c) depict a series of line drawings and an isometric view (FIG. 2(c)) of the heat-shrink band 2 with a large custom-designed flat-top hemi-spherical buckle 4. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area. Note that the custom designed buckle has an insertion peg 5′ that allows the custom designed buckle to be snapped onto the band 2 through a snap on hole, like holes in a regular trousers' belt. Note that this particular buckle has a flat top 7.

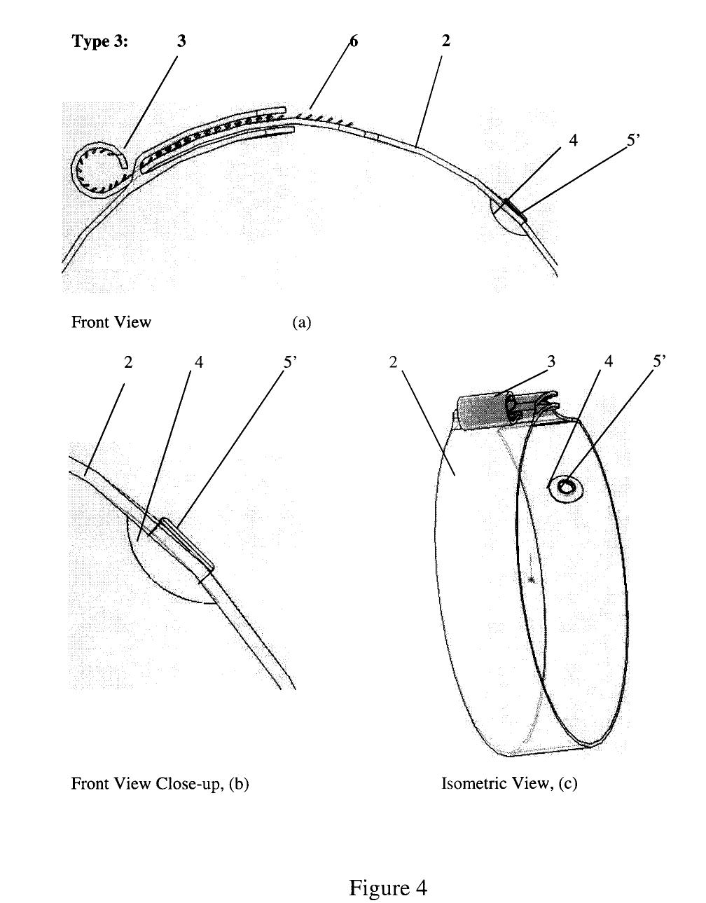

FIGS. 3(a), 3(b) and 3(c) depict a series of line drawings and an isometric view (FIG. 3(c)) of the heat-shrink band 2 with a large custom-designed round-top hemi-spherical buckle 4. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area. Note that the custom designed buckle has an insertion peg 5′ that allows the custom designed buckle to be snapped onto the band 2 through a snap on hole, like holes in a regular trousers' belt. Note that this particular buckle has a round top 7.

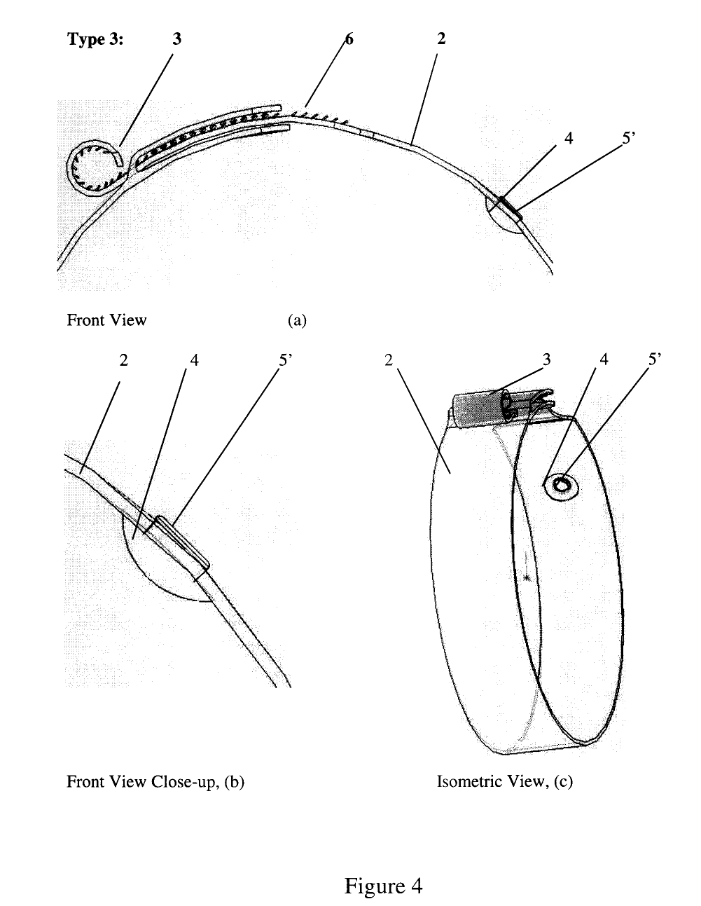

FIGS. 4(a), 4(b) and 4(c) depict a series of line drawings and an isometric view (FIG. 4(c)) of the heat-shrink band 2 with a small custom-designed round-top hemi-spherical buckle 4. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area. Note that the custom designed buckle has an insertion peg 5′ that allows the custom designed buckle to be snapped onto the band 2 through a snap on hole, like holes in a regular trousers' belt. Note that this particular buckle has a round top 7.

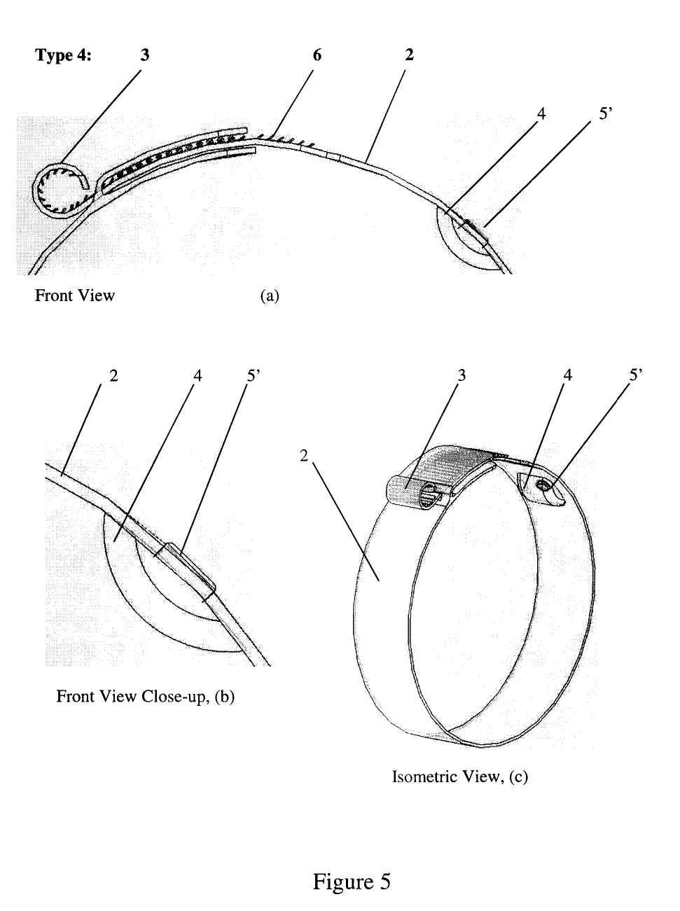

FIGS. 5(a), 5(b) and 5(c) depict a series of line drawings and an isometric view (FIG. 5(c)) of the heat-shrink band 2 with a custom-designed round-top hemi-cylindrical buckle 4. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area. Note that the custom designed buckle has an insertion peg 5′ that allows the custom designed buckle to be snapped onto the band 2 through a snap on hole, like holes in a regular trousers' belt. Note that this particular buckle has a round top 7.

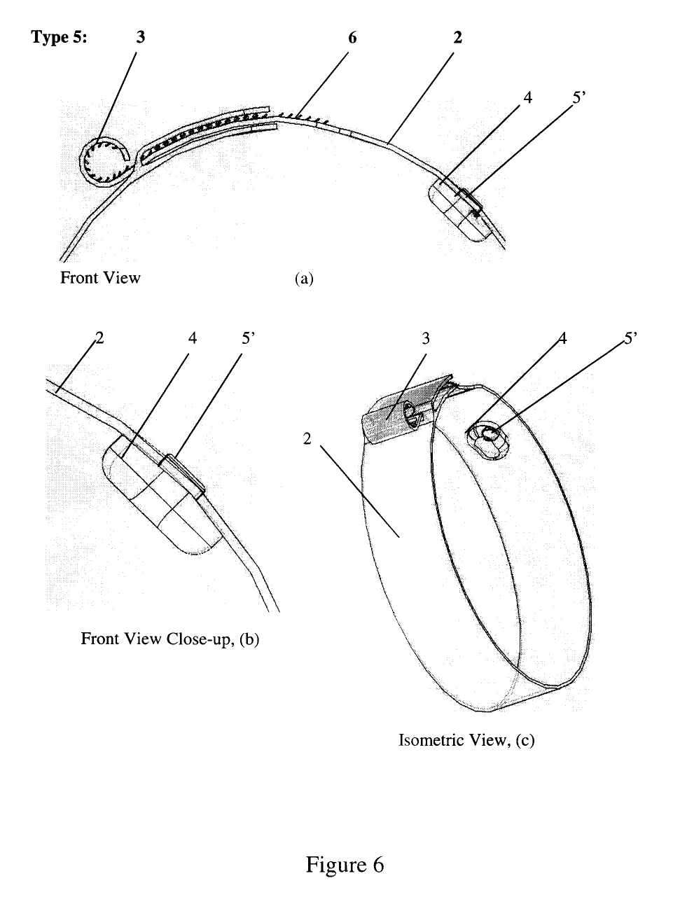

FIG. 6-Heat-shrink band with a flat-top custom-designed oval-shaped buckle FIGS. 6(a), 6(b) and 6(c) depict a series of line drawings and an isometric view (FIG. 6(c)) of the heat-shrink band 2 with an oval-shaped custom-designed flat-top buckle 4. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area. Note that the custom designed buckle has an insertion peg 5′ that allows the custom designed buckle to be snapped onto the band 2 through a snap on hole, like holes in a regular trousers' belt. Note that this particular buckle has a flat top 7.

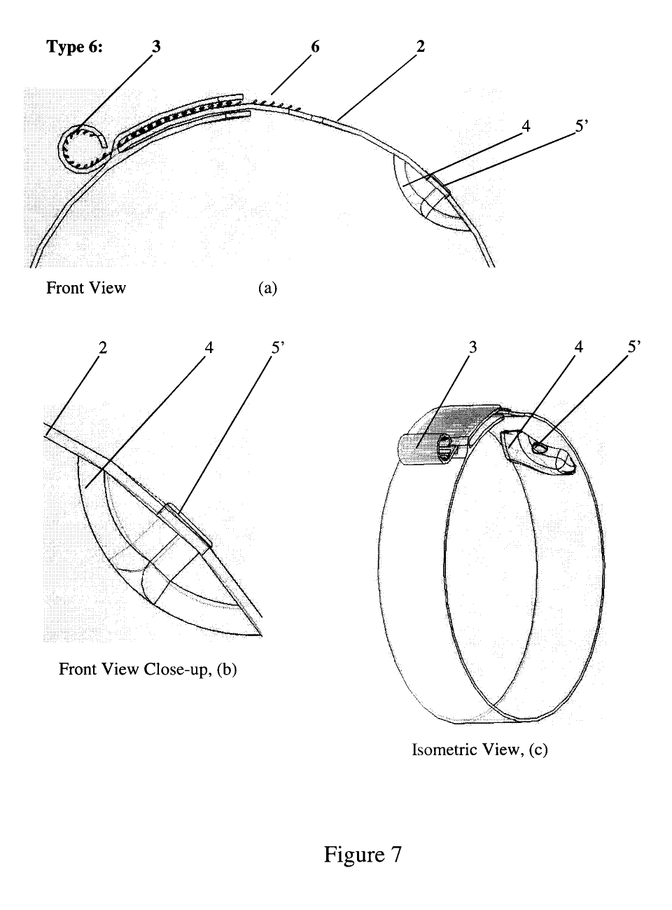

FIG. 7 Heat-shrink band with a custom-designed irregular shape buckle FIGS. 7(a), 7(b) and 7(c) depict a series of line drawings and an isometric view (FIG. 7(c)) of the heat-shrink band 2 with a custom-designed irregular-top irregular shape buckle 4. The actual heat shrink band is denoted by 2 and 3 depicts an attachment mechanism like a belt buckle with self-locking serrated ends 6 to allow a surgeon to place the band in an encircling manner around the sclera with the actual custom-made buckle 4 over the retinal tear area. Note that the custom designed buckle has an insertion peg 5′ that allows the custom designed buckle to be snapped onto the band 2 through a snap on hole, like holes in a regular trousers' belt. Note that this particular buckle has an irregular shape top 7.

The heat-shrink band equipped with a custom-designed buckle implant of the invention is considered to have several advantages over prior art materials and particularly over silicone sponge as currently used in the treatment of retinal detachment. These advantages include the finding that the heat-shrink implant, unlike silicone sponge, does not have macroscopic pores but rather has microscopic pores and hence reduces chances of infection. In addition, the new implant, like the prior art silicone sponge but unlike prior gelatin implants, is nonabsorbable.

It will thus be seen that the heat-shrink band equipped with custom-designed buckles of the present invention provides a combination of a significant number of desired properties, and that the objects set forth above, among those made apparent from the preceding description, are efficiently attained. Since certain changes may be made in the above composition of matter, in the carrying out of the foregoing method of its preparation, and in the resultant implant article as set forth, without departing from the scope of the invention, it is intended that all matter contained in the above description be interpreted as illustrative and not in a limiting sense.

It is also to be understood that the following claims are intended to cover all of the generic and specific features of the invention herein described, and all statements of the scope of the invention which, as a matter of language, might be said to fall there between.

The heat-shrink band equipped with a custom-designed buckle that perfectly fits the geometry of the retinal tear is inserted into Tenon's space, near the detached retinal portion where the retinal holes are located. The band is then heated by means of a hot tip, optical fiber-delivered laser, RF or other conventional means in order to achieve the desired amount of tension in the band and indentation in the sclera. The heat shrinking procedure may be done in stages so that in subsequent visits to the surgeon no open surgery is needed but only transcutaneous heating and shrinking of the band is performed to gradually drain the sub-retinal fluids and perfectly cover the detached area and have the retina reattach to the choroid.

These explanations and other objects of the said invention will become apparent from the study of the drawings and the preceding description of the preferred embodiments of the present invention.

1. A method and an apparatus for correcting retinal detachments by heat-shrinking a specially designed scleral band equipped with a snap-on custom-made buckle for scleral indentation over the retinal tear region.

2. A method and an apparatus of correcting retinal detachments including the steps of claim 1 such that the heat shrink band is made from a heat shrink material such as a polymer and is equipped with a custom-made buckle to perfectly fit the topological geometry of the retinal detachment region over the underlying choroid and the sclera, the conjunctiva and within the Tenon's space wherein the band is heat shrunk to create a perfect buckle over the retinal tear region to reattach the retina to the choroid and cause absorption of the subretinal fluid.

3. A method of correcting retinal detachments through a small conjunctival incision comprising the steps of inserting an heat shrink band into Tenon's space to the depth of the break; using a custom-designed buckle for scleral indentation in the eye; leaving said member in place until subretinal fluid has absorbed and the retina has reattached.

4. A method of correcting retinal detachments including the steps of claim 2, the additional step of leaving said member in place for at least three days after reattachment to promote better adhesion.

5. A method of correcting retinal detachments including the steps of claim 2, the additional step of collapsing said member in stages prior to removing same.

6. A heat shrink band equipped with a custom-designed buckle for scleral indentation over the retinal tear for use in the treatment of retinal detachments comprising: a substantially encircling heat shrink band, a custom designed buckle for scleral indentation over the retinal tear, means of heat shrinking the band in a number of stages to gradually drain out subretinal fluid.

7. A heat shrink buckle band equipped with a custom designed buckle in accordance with claim 2, wherein the custom designed buckle is essentially a hemispherical buckle with a peg in the back for snap on attachment to the band.

8. A heat shrink buckle band equipped with a custom designed buckle in accordance with claim 2, wherein the custom designed buckle is essentially a flat-top hemi-spherical buckle with a peg in the back for snap on attachment to the band.

9. A heat shrink buckle band equipped with a custom designed buckle in accordance with claim 2, wherein the custom designed buckle is essentially a hemi-cylindrical buckle with a peg in the back for snap on attachment to the band.

10. A heat shrink buckle band equipped with a custom designed buckle in accordance with claim 2, wherein the custom designed buckle is essentially a flat-top hemi-cylindrical buckle with a peg in the back for snap on attachment to the band.

11. A heat shrink buckle band equipped with a custom designed buckle in accordance with claim 2, wherein the custom designed buckle is essentially an oval shaped buckle with a peg in the back for snap on attachment to the band.

12. A heat shrink buckle band equipped with a custom designed buckle in accordance with claim 2, wherein the custom designed buckle is essentially a flat-top oval shaped buckle with a peg in the back for snap on attachment to the band.

13. A heat shrink buckle band equipped with a custom designed buckle in accordance with claim 2, wherein the custom designed buckle is essentially a custom-made buckle of irregular shape with a peg in the back for snap on attachment to the band.

14. A heat shrink buckle band equipped with a custom designed buckle in accordance with claim 2, wherein the materials used are biocompatible medical grade.

15. A heat shrink buckle band equipped with a custom designed buckle in accordance with claim 2, wherein the materials used is a silicone synthetic rubber material.

16. A heat shrink buckle band equipped with a custom designed buckle in accordance with claim 2 wherein the band material used is a medical grade heat shrink polymer such as polyurethane or polyolefin.

17. A heat shrink buckle band equipped with a custom designed buckle in accordance with claim 2 wherein the band material used is a medical grade heat shrink alloy such as shape memory alloys or Nitinol.

18. The surgical implant article of claim 2 wherein said heating means is a hot tip instrument.

19. The surgical implant article of claim 2 wherein said heating means is a laser.

20. The surgical implant article of claim 2 wherein said heating means is radio frequency (RF) waves.

21. The surgical implant article of claim 2 wherein said heating means is electricjoule heating in case of a shape memory alloy band.

22. The surgical implant article of claim 2 wherein said heating means is microwave.

23. The surgical implant of claim 2 where is the scleral band itself is not of heat-shrink material and is a biocompatible polymer such as silicone rubber.
